(19) United States
(12) Patent Application Publication
(10) Pub. No.: US 2012/0255565 A1
(43) Pub. Date: Oct. 11, 2012

(54) MEDICAL BACKBOARD

(76) Inventors: Ryan Hursh, Billings, MT (US); Noel Simmons, Lewistown, MT (US)

(21) Appl. No.: 13/082,511

(22) Filed: Apr. 8, 2011

(51) Int. Cl. A61F 5/37; A61G 1/00

(52) U.S. Cl. 128/870; 5/625

(57) ABSTRACT

A medical backboard comprising a front board, a rear board, and one or more polypropylene honeycomb inserts situated between the front and rear board. The front board comprises a head recess, a torso recess, and a foot/heel recess for the insertion of gel pads. The front board comprises a plurality of lateral and longitudinal channels and ridges that increase the strength of the board and decrease the overall weight of the board. The rear board comprises lateral ridges in the neck and ankle areas that increase the strength of the board, and it also comprises a head recess, torso recess and/foot/heel recess that create a ground-contacting surface when the board is placed on the ground.

### BACKGROUND OF THE INVENTION

1. Field of the Invention

The present invention relates generally to the field of medical equipment, and more specifically, to a medical backboard for transporting patients.

2. Description of the Related Art

There are a number of prior art references for medical backboards, but none of these references incorporates the structural aspects of the present invention. The following discussion offers some examples of medical backboard-type inventions but is not intended to be a comprehensive review.

U.S. Pat. No. 5,988,173 (Scruggs, 1999) describes a head and neck immobilization system comprising a rigid head, neck and shoulder support board with strap attachment slot members for securing straps to the board. The invention comprises a forehead securing assembly comprising a flexible plastic forehead gripper and elastic immobilizing straps with metal securing clips. The invention further comprises gel-filled, mallet-shaped cervical spine support bags and an around-the-helmet support comprised of an elongated flexible plastic tube filled with BB-sized foam beads.

U.S. Pat. No. 6,065,165 (Delk et al., 2000) provides a medical backboard apparatus in which the patient is actually sandwiched between the front and rear boards of the backboard apparatus. Apertures in the top board allow the patient's face, abdomen and groin to extend through and/or be visible through the top board. Face cushions, chest cushions and leg cushions are inserted on top of the patient between the patient and the top board.

U.S. Pat. No. 6,848,134 (Schenck, 2005) discloses a pad system for use in connection with a medical backboard. The pad system comprises a base pad that is gel-filled and has a slip-resistant bottom surface that comes into contact with the backboard. Additional gel-filled pads, including first and second scapula pads, a buttocks pad, a heel pad and a headrest pad, are placed into openings on the base pad. The invention further comprises inflatable lumbar and knee pads. The base pad is preferably comprised of gel with a greater hardness than the additional gel-filled pads.

U.S. Pat. No. 6,915,805 (Crutchfield, 2005) describes a molded plastic spine board with padding and stiffening members. The padding comprises a foot/heel pad and a torso pad manufactured from almost incompressible cushion material such as urethane. The board further comprises an upper perimeter-stiffening “L” and a lower perimeter-stiffening “L” that are molded around the outer perimeter of the upper and lower sections of the backboard and that meet to form a perimeter-stiffening channel on the right and left sides of the board. The perimeter-stiffening channel extends inward from the outer edge of the board to just inside the outer perimeter of the hand/strap holds, thereby stiffening the board about the outer perimeter and also providing strength for the hand holds. The lower half of the board comprises V-channel stiffeners that lie underneath a torso pad indention and parallel to the longitudinal axis of the board.

U.S. Pat. No. 7,496,980 (Sharps, 2009) provides an operating table conversion platform that is configured to support a patient in a prone position for back surgery or other medical procedures. The invention comprises a head support member, a torso support member, and a leg support members that are arranged to secured the head, torso and legs, respectively, of the patient when moving the patient from a supine position to a prone position and vice versa. The support members are constructed of layered resilient material, such as foam rubber, gel material, or any other suitable material. The head support member optionally comprises an inflation bladder.

None of the inventions addressed above possesses the structural features of the present invention, namely, the inclusion of strategically placed sheets of polypropylene honeycomb inserts between the front and rear boards to provide superior insulation and energy absorption. This and other structural features of the present invention are described more fully below.

### BRIEF SUMMARY OF THE INVENTION

The present invention is a medical backboard comprising a front board, a rear board, and one or more polypropylene honeycomb inserts situated between the front and rear boards. In a preferred embodiment, the front board comprises a head recess, a torso recess, and a foot/heel recess for the insertion of gel pads. Preferably, the backboard has a perimeter and head and foot areas, and the invention further comprises a plurality of apertures along the perimeter of the backboard that serve as handholds, wherein the apertures in the head area are curved to follow the shape of the board and extend from the top of the head recess to the top center of the front board, and wherein the apertures in the foot area are curved to follow the shape of the board and extend from the middle of the foot/heel recess to the bottom center of the front board.

In a preferred embodiment, the backboard has a perimeter and neck and ankle areas, and the backboard is indented along the perimeter at the neck and ankle areas to facilitate the use of webbing. Preferably, the front board comprises a torso area with a back surface, the back surface of the torso area comprises a plurality of lateral ridges, and the polypropylene honeycomb inserts are placed between the lateral ridges on the back surface of the torso area of the front board. The front board preferably comprises one or more longitudinal channels extending from the head recess to the torso recess and from the torso recess to the foot/heel recess.

In a preferred embodiment, the torso recess has a center axis, and the front board comprises a channel extending longitudinally along the center axis of the torso area. Preferably, the head recess has a perimeter, and the head recess comprises a concave recess that is deeper than the head recess and whose circumference is smaller than the perimeter of the head recess. The torso recess preferably comprises a plurality of lateral channels that create the lateral ridges on the back surface of the torso area of the front board.

In a preferred embodiment, the front and rear boards are comprised of a fiberglass resin. Preferably, the rear board comprises a head recess, a torso recess and a foot/heel recess. The rear board preferably further comprises raised lateral ridges in the neck and ankle areas that are roughly laterally aligned with the indentations on the backboard in the neck and ankle areas.

In a preferred embodiment, the backboard has a perimeter, and the head recess, torso recess and foot/heel recess of the rear board provide a ground-contacting surface on the back surface of the backboard when the backboard is placed on the ground or other surface, thereby raising the perimeter of the backboard.

### BRIEF DESCRIPTION OF THE DRAWINGS

FIG. 1 is a front perspective view of the present invention fully assembled with gel pads.

FIG. 2 is a front perspective view of the present invention fully assembled but without gel pads.

FIG. 3 is a perspective section view of the present invention.

FIG. 4 is an exploded view of the present invention.

FIG. 5 is a front view of the front board of the present invention.

FIG. 6 is a rear view of the front board of the present invention.

FIG. 7 is a front view of the rear board of the present invention.

FIG. 8 is a rear view of the rear board of the present invention.

### REFERENCE NUMBERS

1 Backboard

2 Gel pad

3 Aperture/handhold

3A Aperture/handhold above head area of backboard

3B Aperture/handhold beneath foot area of backboard

4 Peg

5A Gel pad recess on front board (head area)

5B Gel pad recess on front board (torso area)

5C Gel pad recess on front board (foot/heel area)

5D Circular recess (in head area)

6 Front board

7 Rear board

8 Polypropylene honeycomb insert

9 Longitudinal channel

10 Lateral channel

11 Longitudinal ridge

12 Lateral ridge

13A Rear board recess (head area)

13B Rear board recess (torso area)

13C Rear board recess (foot/heel)

14A Lateral ridge (neck area)

14B Lateral ridge (top of foot/heel area)

### DETAILED DESCRIPTION OF INVENTION

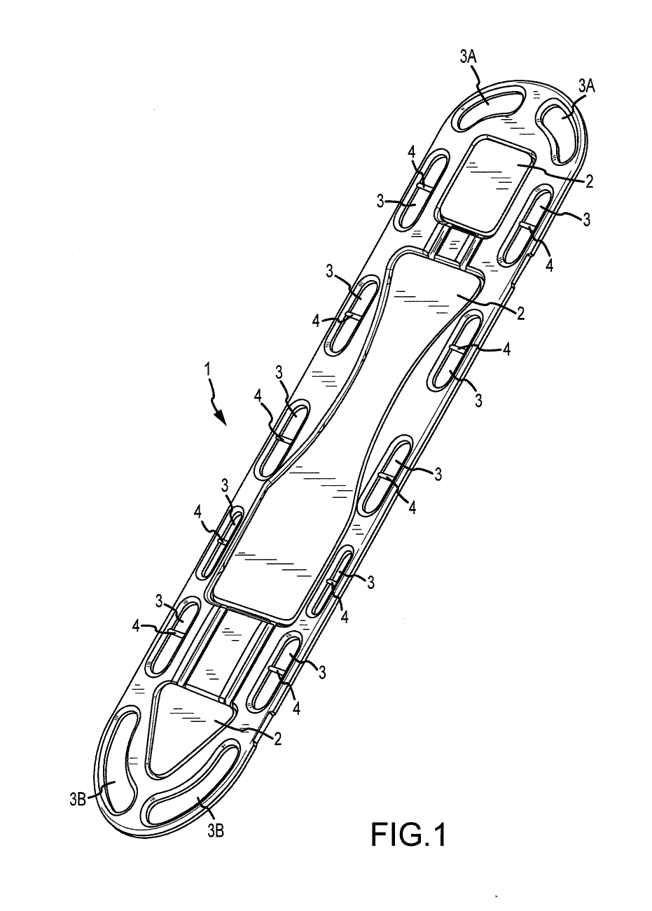

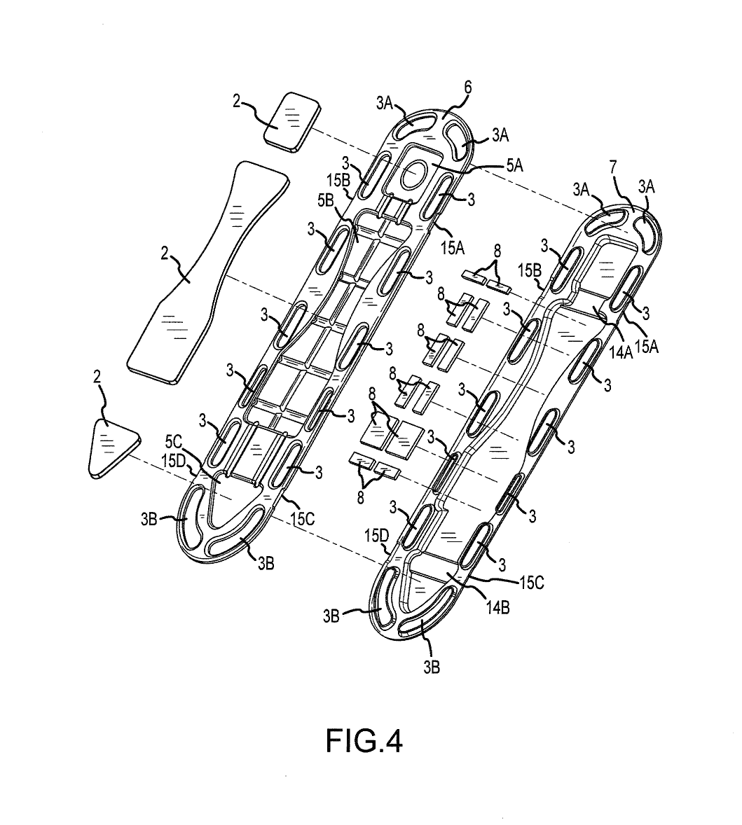

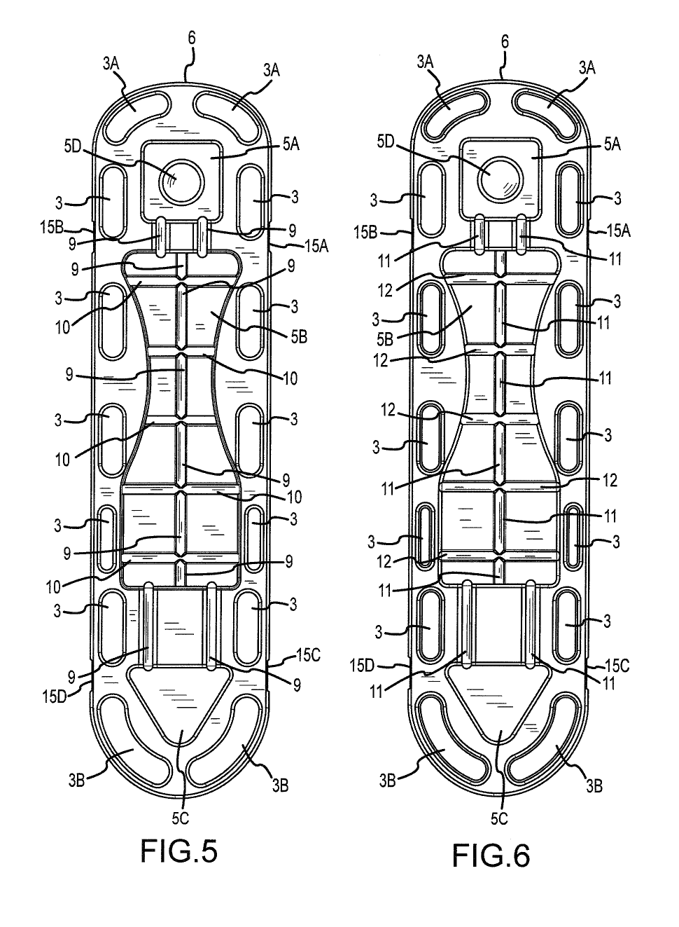

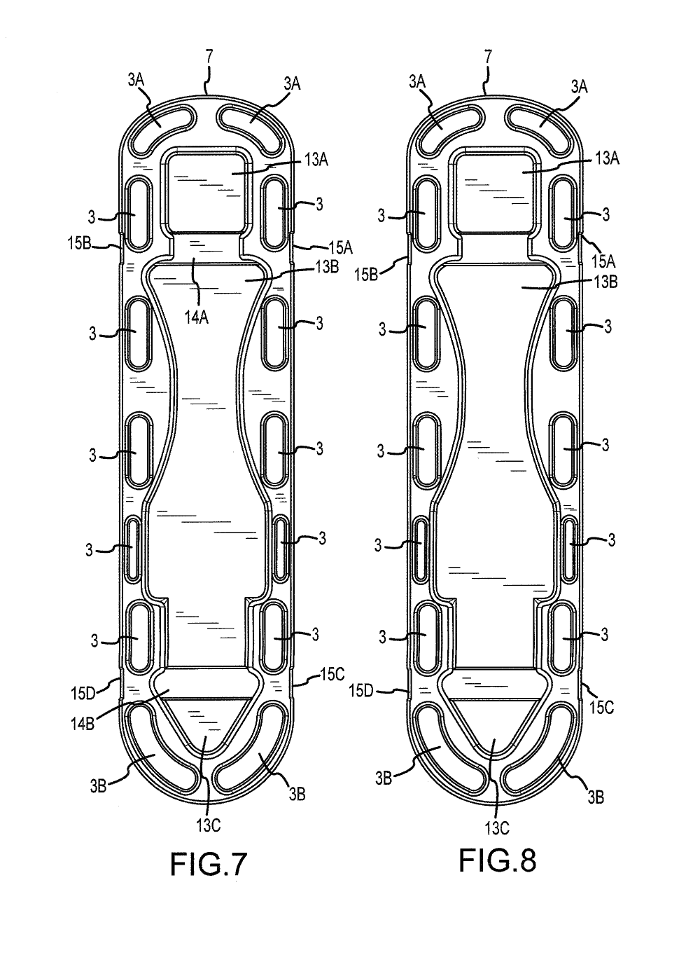

In a preferred embodiment, the backboard 1 is molded in two parts—a front board (see FIGS. 5 and 6) and a rear board (see FIGS. 7 and 8), which are heat sealed together under vacuum press with polypropylene honeycomb inserts (see FIG. 4) in between them. FIG. 1 shows the front and rear boards sealed together to form the backboard 1; the polypropylene honeycomb inserts are not visible because they are inside the backboard (between the front and rear boards).

In a preferred embodiment, gel pads 2 for the head, torso and heel areas are fastened onto the front surface of the front board with hook-and-loop fasteners (on the underside of the gel pads and on the front surface of the front board). In a preferred embodiment, the gel pads 2 may be removed from the front surface of the front board, washed, and reinstalled without having to reapply the adhesive. The gel pads 2, which provide added comfort and stability for the patient, are preferably comprised of SHOCKTEC® gel made by Kemmler Products, Inc. of Mooresville, N.C.

As shown in FIG. 1, the backboard 1 preferably comprises a plurality of apertures 3 along the perimeter of the backboard that serve as handholds. The apertures 3A in the head area and the apertures 3B in the foot area are elongated to meet military specifications and increase ease of handling of the board with a patient on it. The apertures 3A in the head area are curved to follow the shape of the board and extend from the top of the head area gel pad 2 to the top center of the front board. The apertures 3B in the foot area are also curved to follow the shape of the board and extend from the middle of the heel area gel pad 2 to the bottom center of the front board.

Note that all apertures 3 except the apertures 3A, 3B comprise pegs 4 that extend laterally through the center of each aperture 3. The purpose of these pegs 4 is to allow strapping (not shown) to be clipped or fastened to the backboard. Each peg is preferably a ¼-inch dowel comprised of polypropylene. In addition, the backboard 1 is preferably indented (see reference numbers 15A, 15B, 15C and 15D in FIGS. 4-8) at the neck and ankle areas to facilitate the use of webbing (that would run laterally underneath and around the entire backboard) in these areas.

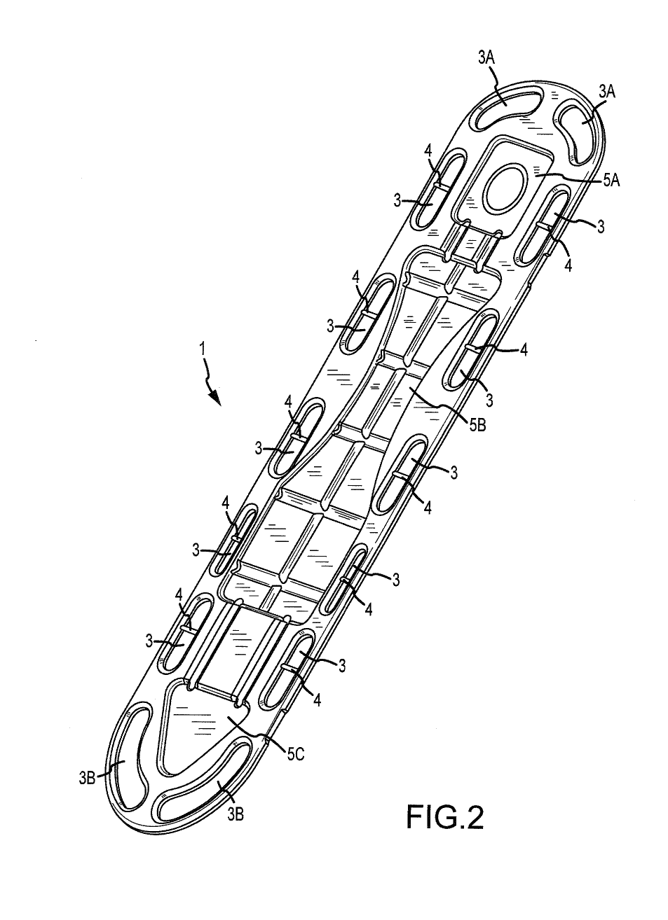

FIG. 2 is a front perspective view of the present invention fully assembled but without gel pads. As shown in this figure, the front surface of the front board comprises recesses in the head 5A, torso 5B and foot/heel areas 5C for the gel pads 2 (not shown). The front board also comprises longitudinal and lateral recesses and ridges, which are discussed further in connection with FIGS. 5 and 6.

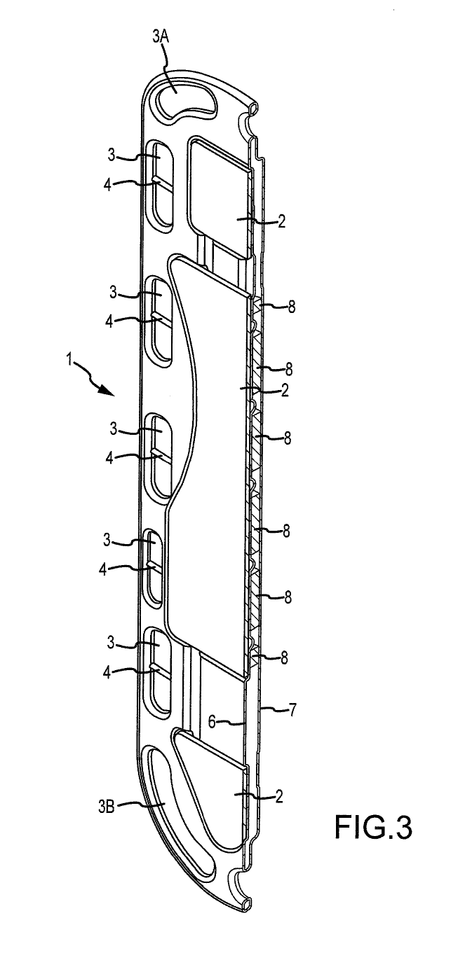

FIG. 3 is a side section view of the present invention. This figure shows the polypropylene honeycomb inserts 8 in the center of the backboard 1, discussed more fully below in connection with FIG. 4.

FIG. 4 is an exploded view of the present invention. As shown in this figure, the backboard 1 comprises a front board 6 and a rear board 7. The pegs 4 (shown in FIG. 1) have been omitted for clarity. This figure also shows the polypropylene honeycomb inserts 8, which are preferably in the form of sheets or blocks, that are placed in between the front and rear boards 6, 7. Although the present invention is not limited to any particular size, shape or number of polypropylene honeycomb inserts 8, in a preferred embodiment, the polypropylene honeycomb inserts 8 are sized and shaped so that they fit in between the ridges 11, 12 in the torso area on the back surface of the front board (see FIG. 6). Note that the height of each polypropylene honeycomb insert may not exceed the combined depth of the recess into which it fits on the back surface of the front board (see FIG. 6) and the recess into which it fits on the front surface of the rear board (see FIG. 7). In a preferred embodiment, the recesses on the back surface of the front board and on the front surface of the rear board are approximately ¼ inches in depth, and the height of each polypropylene honeycomb insert is ½ inch.

The front and rear boards 6, 7 may be molded to accommodate different sizes of polypropylene honeycomb inserts by increasing or decreasing the depth of the recesses on the back surface of the front board and/or on the front surface of the rear board. In a preferred embodiment, the height of the polypropylene honeycomb insert is between ⅜ inch and six inches, and both sides of the polypropylene honeycomb inserts are adhered to the front and rear boards. In a preferred embodiment, the polypropylene honeycomb inserts are adhered to the back surface of the front board 6 and to the front surface of the rear board 7 with an adhesive. In one embodiment, the same fiberglass resin material that is used to form the front and rear boards is used to adhere the polypropylene honeycomb inserts to the front and rear boards; this adhesive need only hold the polypropylene honeycomb inserts in place before the two boards (front and rear) are sealed together under vacuum press.

The polypropylene honeycomb material used in the inserts of the present invention is currently manufactured by Plascore Incorporated of Zeeland, Mich. Technical specifications for this material are attached hereto as Exhibit A and incorporated herein by reference. As used in the present invention, the polypropylene honeycomb inserts provide superior insulation to that of trapped air and/or foam, and they also provide high energy absorption. The backboard of the present invention, with the polypropylene honeycomb inserts, can carry a load of up to 1000 pounds and withstand torque of up to 2000 foot-pounds, which makes it far stronger than existing medical backboards. In addition, the polypropylene honeycomb inserts provide positive buoyancy, thereby increasing the overall buoyancy of the backboard. The polypropylene honeycomb inserts are also radio-translucent, which means they will not interfere with MRI and CAT scans.

FIG. 5 is a front view of the front board of the present invention. (Note that the pegs 4 are also omitted from FIGS. 5-8 for clarity.) This figure shows not only the gel pad recesses 5A, 5B and 5C in the front surface of the front board 6 but also the channels 9 extending longitudinally from the head area 5A to the torso area 5B, along the center axis of the torso area 5B, and from the torso area 5B to the foot/heel area 5C. The gel pad recess 5A in the head area preferably comprises a circular and concave recess 5D that is deeper than the gel pad recess 5A and whose circumference is smaller than the perimeter of the gel pad recess 5A. The purpose of this additional recess 5D is to accommodate the back of a patient's head. FIG. 5 also shows the channels 10 extending laterally across the torso area 5B. Note that the longitudinal and lateral channels 9, 10 on the front surface of the front board 6 create correspondingly placed longitudinal and lateral ridges 11, 12, respectively, on the back surface of the front board (see FIG. 6).

The purpose of the longitudinal and lateral channels 9, 10 is two-fold. First, the inclusion of (i) longitudinal channels connecting the gel pad recesses 5A, 5B, 5C to one another, (ii) an essentially continuous longitudinal channel down the center (or “spine”) of the board, and (iii) a plurality of lateral channels in the torso area 5B, all serve to increase the overall strength and integrity of the front board 6 so that it can withstand greater weight and torque. Second, these channels, together with the gel pad recesses 5A, 5B and 5C, decrease the overall weight of the backboard as compared to a solid board with no recesses or channels. (Note that although recesses 5A, 5B, 5C and 5D are labeled on FIG. 6, they are actually protrusions, not recesses, on FIG. 6 because they are the reverse side of the recesses shown in FIG. 5.)

FIG. 7 is a front view of the rear board of the present invention, and FIG. 8 is a rear view of the rear board of the present invention. In a preferred embodiment, both the front 6 and rear 7 boards are comprised of a molded fiberglass resin. As shown in these figures, the front surface of the rear board comprises recesses in the head 13A, torso 13B and foot/heel 13C areas. Note also that the rear board preferably comprises a raised lateral ridge 14A in the neck area of the front surface of the board, between the head recess 13A and the torso recess 13B. This raised lateral ridge 14A is thickened as compared to the head and torso recesses 13A, 13B. The purpose of this raised lateral ridge 14A is to provide added strength in the rear board 7 at the same lateral position as the indentations 15A, 15B in the rear board. Similarly, the rear board 7 preferably comprises a raised lateral ridge 14B at the top of the foot/heel area 13C on the front surface of the board to provide added strength at the same lateral position as the indentations 15C, 15D in the rear board.

Although the torso area recess 13B is necessary to accommodate the polypropylene inserts 8, all three recesses serve the purpose of providing a ground-contacting surface on the back surface of the backboard 1 when it is placed on the ground or other surface. Thus, the recesses 13A, 13B and 13C on the front surface of the rear board 7 create protrusions 13A, 13B and 13C on the back surface of the rear board (see FIG. 8). These protrusions 13A, 13B and 13C serve to raise the outer perimeter of the backboard (including the handholds 3, 3A and 3B) so that the handholds are off the ground when the backboard is lying on the ground. This feature makes it easier for the backboard to be lifted with a patient or other load on it.

The present invention is not limited to any particular size of the front 6 and rear 7 boards; the entire backboard may be sized to accommodate different types of patients, including, but not limited to, bariatric and pediatric patients. In the current embodiment, the backboard 1 is approximately seventy-two (72) inches long, approximately eighteen (18) inches wide, and approximately one inch thick.

Although the preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

We claim:

1. A medical backboard comprising a front board, a rear board, and one or more polypropylene honeycomb inserts situated between the front and rear boards.

2. The medical backboard of claim 1, wherein the front board comprises a head recess, a torso recess, and a foot/heel recess for the insertion of gel pads.

3. The medical backboard of claim 2, wherein the backboard has a perimeter and head and foot areas, and further comprising a plurality of apertures along the perimeter of the backboard that serve as handholds, wherein the apertures in the head area are curved to follow the shape of the board and extend from the top of the head recess to the top center of the front board, and wherein the apertures in the foot area are curved to follow the shape of the board and extend from the middle of the foot/heel recess to the bottom center of the front board.

4. The medical backboard of claim 2, wherein the backboard has a perimeter and neck and ankle areas, and wherein the backboard is indented along the perimeter at the neck and ankle areas to facilitate the use of webbing.

5. The medical backboard of claim 1, wherein the front board comprises a torso area with a back surface, wherein the back surface of the torso area comprises a plurality of lateral ridges, and wherein the polypropylene honeycomb inserts are placed between the lateral ridges on the back surface of the torso area of the front board.

6. The medical backboard of claim 2, wherein the front board comprises one or more longitudinal channels extending from the head recess to the torso recess and from the torso recess to the foot/heel recess.

7. The medical backboard of claim 2, wherein the torso recess has a center axis, and wherein the front board comprises a channel extending longitudinally along the center axis of the torso area.

8. The medical backboard of claim 2, wherein the head recess has a perimeter, and wherein the head recess comprises a concave recess that is deeper than the head recess and whose circumference is smaller than the perimeter of the head recess.

9. The medical backboard of claim 5, wherein the torso recess comprises a plurality of lateral channels that create the lateral ridges on the back surface of the torso area of the front board.

10. The medical backboard of claim 1, wherein the front and rear boards are comprised of a fiberglass resin.

11. The medical backboard of claim 2, wherein the rear board comprises a head recess, a torso recess and a foot/heel recess.

12. The medical backboard of claim 4, wherein the rear board further comprises raised lateral ridges in the neck and ankle areas that are roughly laterally aligned with the indentations on the backboard in the neck and ankle areas.

13. The medical backboard of claim 11, wherein the backboard has a perimeter, and wherein the head recess, torso recess and foot/heel recess of the rear board provide a ground-contacting surface on the back surface of the backboard when the backboard is placed on the ground or other surface, thereby raising the perimeter of the backboard.
